(19) United States
(12) Patent Application Publication
(10) Pub. No.: US 2025/0065020 A1
(43) Pub. Date: Feb. 27, 2025

(54) BIOCOMPATIBLE METAL ION-CHITOSAN HYDROGELS WITH ANTIMICROBIAL PROPERTIES

(71) Applicant: FLORIDA ATLANTIC UNIVERSITY BOARD OF TRUSTEES, Boca Raton, FL (US)

(72) Inventors: Vivian MERK, Boca Raton, FL (US); Satviki SINGH, Boca Raton, FL (US); Saniela SCHEURLE, Boca Raton, FL (US)

(21) Appl. No.: 18/727,820

(22) PCT Filed: Jan. 11, 2023
(86) PCT No.: PCT/US23/60457

§ 371 Date: Jul. 10, 2024

Related U.S. Application Data
(60) Provisional application No. 63/298,469, filed on Jan. 11, 2022.

(51) Int. Cl. A61L 28/00; A61L 15/28; A61L 15/42

(52) U.S. Cl. CPC A61L 28/008; A61L 15/28; A61L 15/42; A61L 2300/104; A61L 2300/406

(57) ABSTRACT

Disclosed herein are chitosan hydrogels including one or more metal ions. The hydrogels have antimicrobial properties and can be used to treat wounds and other openings in the skin.

### CROSS-REFERENCE TO RELATED APPLICATION

This application claims the benefit of U.S. Provisional Application 63/298,469, filed Jan. 11, 2022, the contents of which are hereby incorporated by reference in its entirety.

### FIELD OF THE INVENTION

This invention relates to wound dressings and more particularly to chitosan hydrogels with antimicrobial properties for use in treating wounds.

### BACKGROUND

In the United States, nosocomial infections (NI) affect nearly 1.7 million people across the country per year. NIs are infections patients contract in medical settings like hospitals and clinics. 1 in every 31 hospital patients in the US is affected by a NI. The situation is worse for developing countries due to the lack of resources to prevent, identify, and treat such infections. These infections result in illnesses, health complications, and elongated hospital stays, thus resulting in higher medical costs. In extreme cases, NIs can even result in death. NIs are usually caused by bacteria, most commonly S. aureus, P. aeruginosa, and E. coli strains, which often arise as skin infections from topical wounds.

Wound dressings play a significant role in preventing and treating such complications because they can be directly applied to wounds and other openings that could potentially lead to NIs. Hydrogels are an effective form of wound dressing because they create a moisture barrier for the wound, absorb wound exudates, create an acidic pH around the wound, encourage wound healing, protect the wound from bacterial infection, and/or promote the regeneration of healthy tissue.

Chitosan is a non-toxic, biocompatible, and readily available biomaterial, rendering it ideal for localized drug delivery. Chitosan is degraded by human enzymatic activities, in particular lysozymes, rendering it biodegradable. It is known to promote wound healing, due to its bacteriostatic properties. At physiological pH, chitosan is positively charged, which promotes bioadhesion and increases retention at an application site.

There remains a need for improved wound dressings. There remains a need for improved systems and methods for treating and preventing bacterial infections, including nosocomial infections. There remains a need for improved systems and methods for treating infections caused by S. aureus, P. aeruginosa, and/or E. coli. There remains a need for improved chitosan hydrogels for drug delivery and wound treatment. There remains a need for improved biocompatible metal ion-chitosan hydrogels with antimicrobial properties to encourage wound healing and protect wounds from bacterial infections. There remains a need for chitosan hydrogels with improved degradation and toxicity profiles.

### BRIEF DESCRIPTION OF THE FIGURES

FIG. 1 depicts a schematic of an experimental set up for the preparation of chitosan hydrogels.

FIG. 2 depicts a graph showing FT-IR spectroscopy of Ag⁺-containing chitosan hydrogels.

FIG. 3A depicts a scanning electron micrograph of a silver-containing chitosan hydrogel prepared using 0.1 wt. % AgNO₃.

FIG. 3B depicts scanning electron micrograph of a silver-containing chitosan hydrogel prepared using 0.3 wt. % AgNO₃.

FIG. 4A depicts results from Kirby-Bauer tests of silver-containing hydrogels against P. Aeruginosa.

FIG. 4B depicts results from Kirby-Bauer tests of silver-containing hydrogels against E. coli.

FIG. 5 depicts a graph showing the standard zones of inhibition that resulted from the antibacterial tests of the hydrogels [0.1 wt. %, 0.2 wt. %, and 0.3 wt. %, AgNO₃] against S. aureus. ANOVA, Tukey post hoc test, p=0.033, F=13.153.

FIG. 6A depicts a graph showing the swelling ratio of silver-containing hydrogels soaked in deionized water. Swelling data was averaged over 8 samples.

FIG. 6B depicts a graph showing the swelling ratio of silver-containing hydrogels soaked in ethylene glycol.

FIG. 7 depicts a graph showing the zones of inhibition that resulted from the antibiotic absorption and release test of the hydrogels [0.1 wt. %, 0.2 wt. %, and 0.3 wt. %, AgNO₃] against S. aureus. ANOVA, Tukey post hoc test, p>0.05 for all [AgNO₃] comparisons.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are biocompatible metal ion-chitosan hydrogels for treating wounds that include chitosan in combination with a metal ion selected from silver ions, copper ions, or a combination thereof. In some implementations, the hydrogel includes chitosan and silver ions, while in some implementations the hydrogel includes chitosan and copper ions. In further implementations the hydrogel includes a mixture of silver and copper ions.

The hydrogels have antimicrobial properties and thus can be used to treat and protect wounds. In some implementations the hydrogels can be used to treat and/or prevent bacterial infections. In some implementations the hydrogels can be used to treat and/or prevent S. aureus infections. In some implementations, the hydrogel can include an antimicrobial peptide, for example cyclic lipopeptide-4 (CLP-4), which is useful against gram-positive bacterial biofilm formations. It was found that both the compatibility of the hydrogel with CLP-4 and the effectiveness of the hydrogel against S. aureus increases with the concentration of silver, which is associated with lower surface porosity of the hydrogel, indicating that an increase in silver concentration and a decrease in surface porosity optimizes CLP-4 drug delivery and anti-biofilm activity. In some implementations the hydrogel is biodegradable and can be implanted into a subject. In some implementations, the hydrogels can be used to deliver CLP-4 to a target location within a subject.

In some implementations, the disclosed hydrogels can be obtained by combining a chitosan with one or more metal ions to form the hydrogel

In some implementations, the hydrogels contain chitosan and metal ions at a (combined) concentration from 0.1-25 wt. %, from 0.1-10 wt. %, from 0.1-5 wt. %, from 0.5-10 wt. %, from 0.5-5 wt. %, from 0.5-2.5 wt. %, from 1-10 wt. %, from 1-8 wt. %, from 1-6 wt. %, from 1-5 wt. %, from 1-2.5 wt. %, from 2-10 wt. %, from 2-8 wt. %, from 2-6 wt. %, from 2-5 wt. %, from 2-4 wt. %, or from 2-3 wt. %. In some implementations the hydrogels include water at a concentration from 75-99.9 wt. %, from 90-99.9 wt. %, from 95-99.9 wt. %, from 90-99.5 wt. %, from 95-99.5 wt. %, from 97.5-99.5 wt. %, from 90-99 wt. %, from 92-99 wt. %, from 94-99 wt. %, from 95-99 wt. %, from 97.5-99 wt. %, from 90-98 wt. %, from 92-98 wt. %, from 94-98 wt. %, from 95-98 wt. %, from 96-98 wt. %, from 97-98 wt. %, from 93-97 wt. %, or from 95-97 wt. %.

Chitosan is a polysaccharide including D-glucosamine units. It is commonly obtained by alkaline hydrolysis of the N-acetyl groups of chitin. In certain implementations, the chitosan is at least 60% deacylated (i.e., no more than 40% N-acylated), at least 65% deacylated (i.e., no more than 35% N-acylated), at least 70% deacylated (i.e., no more than 30% N-acylated), at least 75% deacylated (i.e., no more than 25% N-acylated), at least 80% deacylated (i.e., no more than 20% N-acylated), at least 85% deacylated (i.e., no more than 15% N-acylated), at least 90% deacylated (i.e., no more than 10% N-acylated), at least 95% deacylated (i.e., no more than 5% N-acylated), or at least 98% deacylated (i.e., no more than 2% N-acylated). In certain implementations, the chitosan can have a degree of deacylation from 60%-98%, from 60%-90%, from 60%-80%, from 60%-70%, from 70%-98%, from 70%-90%, from 70%-80%, 80%-98%, from 80%-90%, or from 90%-98%. In certain implementations, the chitosan can have a degree of N-acylation from 2-40%, from 10%-40%, from 20%-40%, from 30%-40%, from 20%-30%, from 10%-30%, from 5%-30%, from 10%-20%, from 5%-20% from 5%-10%, or from 2-5%.

In certain implementations, the chitosan in the hydrogels has an average molecular weight from 500-50,000 Da, from 500-25,000 Da, 500-10,000 Da, from 500-5,000 Da, 500-2,500 Da, from 1,000-25,000 Da, from 1,000-15,000 Da, from 1,000-10,000 Da, from 1,000-5,000 Da, from 1,000-2,500 Da, from 1,500-25,000 Da, from 1,500-10,000 Da, from 1,500-5,000 Da, from 2,500-5,000 Da, from 5,000-15,000, from 5,000-10,000, or from 10,000-25,000 Da.

The disclosed hydrogels can include one or more antimicrobial metal ions. In some implementations, the hydrogels can include one or more of silver ions, copper ions, zinc ions, cobalt ions, iron ions, manganese ions, or nickel ions. In some implementations, the hydrogels include silver ions as the sole antimicrobial metal. In some implementations, the hydrogels include copper ions as the sole antimicrobial metal. In some implementations, the hydrogels include silver and copper ions as the sole antimicrobial metals. The metal ions may be provided in the hydrogel at a concentration from 0.001-1 wt. %, from 0.01-1 wt. %, from 0.1-1 wt. %, from 0.001-0.01 wt. %, from 0.01-0.1 wt. %, from 0.025-0.5 wt. %, from 0.05-0.25 wt. %, from 0.025-0.25 wt. %, from 0.05-0.5 wt. %, from 0.1-0.5 wt. %, or from 0.5-1 wt. %. Unless specified to the contrary, in reference to metal and metal ions wt. % refers to the metal ion alone, not the wt. % of the metal salt used to prepare the hydrogel.

In some implementations, the hydrogel will include excess chitosan relative to metal ions. In some implementations, the hydrogels can include chitosan and metal ion in a weight ratio (chitosan:metal ions) from 100:1 to 1:1, from 50:1 to 1:1, from 25:1 to 1:1, from 10:1 to 1:1, from 100:1 to 50:1, from 75:1 to 25:1, from 50:1 to 25:1, from 50:1 to 40:1, from 40:1 to 10:1, from 40:1 to 25:1, from 40:1 to 20:1, from 20:1 to 5:1, or from 30:1 to 5:1.

In some implementations the chitosan hydrogel includes silver ions at a concentration from 0.001-1 wt. %, from 0.01-1 wt. %, from 0.1-1 wt. %, from 0.001-0.01 wt. %, from 0.01-0.1 wt. %, from 0.025-0.5 wt. %, from 0.05-0.25 wt. %, from 0.025-0.25 wt. %, from 0.05-0.5 wt. %, from 0.1-0.5 wt. %, or from 0.5-1 wt. %.

In some implementations, hydrogels includes chitosan and silver ions in a weight ratio (chitosan:silver ions) from 100:1 to 1:1, from 50:1 to 1:1, from 25:1 to 1:1, from 10:1 to 1:1, from 100:1 to 50:1, from 75:1 to 25:1, from 50:1 to 25:1, from 50:1 to 40:1, from 40:1 to 10:1, from 40:1 to 25:1, from 40:1 to 20:1, from 20:1 to 5:1, or from 30:1 to 5:1.

In some implementations the chitosan hydrogel includes copper ions at a concentration from 0.001-1 wt. %, from 0.01-1 wt. %, from 0.1-1 wt. %, from 0.001-0.01 wt. %, from 0.01-0.1 wt. %, from 0.025-0.5 wt. %, from 0.05-0.25 wt. %, from 0.025-0.25 wt. %, from 0.05-0.5 wt. %, from 0.1-0.5 wt. %, or from 0.5-1 wt. %.

In some implementations, hydrogels can include chitosan and copper ions in a weight ratio (chitosan:copper ions) from 100:1 to 1:1, from 50:1 to 1:1, from 25:1 to 1:1, from 10:1 to 1:1, from 100:1 to 50:1, from 75:1 to 25:1, from 50:1 to 25:1, from 50:1 to 40:1, from 40:1 to 10:1, from 40:1 to 25:1, from 40:1 to 20:1, from 20:1 to 5:1, or from 30:1 to 5:1.

In some implementations the chitosan hydrogel includes a combination of silver and copper ions at a concentration from 0.001-1 wt. %, from 0.01-1 wt. %, from 0.1-1 wt. %, from 0.001-0.01 wt. %, from 0.01-0.1 wt. %, from 0.025-0.5 wt. %, from 0.05-0.25 wt. %, from 0.025-0.25 wt. %, from 0.05-0.5 wt. %, from 0.1-0.5 wt. %, or from 0.5-1 wt. %.

In some implementations, hydrogels can include chitosan and copper silver ions in a weight ratio (chitosan:silver+copper ions) from 100:1 to 1:1, from 50:1 to 1:1, from 25:1 to 1:1, from 10:1 to 1:1, from 100:1 to 50:1, from 75:1 to 25:1, from 50:1 to 25:1, from 50:1 to 40:1, from 40:1 to 10:1, from 40:1 to 25:1, from 40:1 to 20:1, from 20:1 to 5:1, or from 30:1 to 5:1.

In some implementations, the chitosan hydrogels include one or more additional therapeutic agents, for example analgesics, anti-inflammatories, and/or additional antibiotics.

In some implementations, the chitosan hydrogels include one or more analgesics, such as opioids (for example fentanyl, morphine, codeine, oxycodone, hydrocodone, oxymorphone, hydromorphone), capsaicin, diclofenac, lidocaine, benzocaine, methyl salicylate, trolamine, prilocaine, pramoxine, dibucaine, phenol, tetracaine, camphor, dyclonine, and menthol. In some implementations, the chitosan hydrogels include one or more anti-inflammatories. Suitable anti-inflammatories include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, and zomepirac sodium In some implementations, the chitosan hydrogel can include one or more antibiotics such as β-lactam-containing antibiotics (which includes penams, cephalosporins, cephamycins, monobactams, carbapenams, and carbocephems), quinolones (which includes fluoroquinolones), aminoglycosides, monobactams, carbapenems, tetracyclines, macrolides, antimicrobial peptides (which includes glycopeptides and lipopeptides), and others. In some implementations, the chitosan hydrogel can include one or more penicillin antibiotics. In some implementations, the chitosan hydrogel can include one or more tetracycline antibiotics.

In some embodiments, the chitosan hydrogel can include one or more compounds selected from penicillin G, anroxicillin, nafcillin, oxacillin, dicloxacillin, flucloxacillin, ampicillin, carbenicillin, ticarcillin, piperacillin, chloramphenicol, streptomycin, neomycin, kanamycin, amikacin, gentamycin, tobramycin, sisonicin, arbekacin, apramycin, netilmicin, paromomycin, spectinonycin, ciprofloxacin, levofloxacin, lomefloxacin, moxifloxacin, norfloxacin, ofloxacin, sparfloxacin, trvafloxacin, gatifloxacin, genifloxacin, cinoxacin, nalidixic acid, azithromycin, clarithromycin, dirithromycin, erythromycin, roxithromycin, troleandomycin, telithromycin, spectinomycin, indolicidin, defensin, cecropin, magainin, vancomycin, teicoplanin, telavancin, ramoplanin, decaplanin, bleomycin, colistin (polymyxin E), colistin A (polymyxin E1), colistin B (polymyxin E2), colistin sulfate, colistimethate sodium, actinomycin, bacitracin, polymyxin B, gentarnicin, gentamicin sulfate, neomycin, kananycin, tobramycin, metronidazole, clotrimazole, secnidazole, ornidazole, tinidazole, linezolid, doxycycline, tetracycline, oxytetracycline, chlortetracycline, demeciocycline, lymecycline, meclocycline, methacycline, ninocycline, rolitetracycline, and tigecycline. In some implementations, the chitosan hydrogels include one or more of ampicillin, tetracycline, and chloramphenicol.

In some implementations, the chitosan hydrogel can include one or more antimicrobial peptides (including glycopeptides and lipopeptides). In some implementations, the chitosan hydrogel can include one or more of gramicidin, polymyxin, daptomycin, colistin, vancomycin, teicoplanin, telavancin, ramoplanin, decaplanin, corbomycin, complestatin, oritavancin, surfactin, iturin, fengycin, tensin, chromobactomycin, or dalbavancin. In some implementations, the chitosan hydrogel can include cyclic lipopeptide-4 (“CLP-4”).

Also disclosed are methods of treating a wound by administering the chitosan hydrogel to the site the wound. As used herein, a wound refers to a tissue injury or damage, using involving laceration, breaking, or disrupting of tissue. A wound may be an internal wound or an external wound. An external wound is a wound to skin tissue and is typically observable via visual inspection. External wounds can be accompanied by internal wound. Internal wounds involve damage or injury to tissue types other that the external skin. Exemplary wounds that may be treated with the chitosan hydrogels include a trauma wound, a surgical wound, a burn wound, or an ulcer wound. The chitosan hydrogels can be used to treat ulcer wounds caused by infection and/or blood flow disruptions (for example diabetes, atherosclerosis, pressure, venous insufficiency, and the like). Wound patients with coagulation disorders may be advantageously treated with the chitosan hydrogels, for instance, diabetics, hemophiliacs, patients with vitamin K deficiency, Von Willebrand disease or other clotting factor deficiencies. The chitosan hydrogels can be used to treat wounds in patients undergoing anti-coagulation therapy, for instance patients receiving heparin, fandaparinux, idraparinux, vitamin K, coumadin, direct thrombin inhibitors like argatroban, dabigatran, factor Xa inhibitors like rivaroxaban, apixaban and edoxaban, anti-platelet agents such as clopidogrel and prasugrel. The chitosan hydrogels can be used to treat wounds arising from medical procedures such as percutaneous coronary intervention, stent and/or valve placement or repair, transfusion, and dialysis. In some implementations the chitosan hydrogels can be used to treat internal wounds, for instance internal wounds caused by surgery, infection, blood flow disruptions, or trauma.

The chitosan hydrogels can be included in a wide variety of pharmaceutical compositions, for instance those including pharmaceutically acceptable carriers. Suitable carriers include water, saline and other liquid formulations, which can be directly administered to a wound site or injected into a patient. In such cases the chitosan hydrogels can be administered by syringe, dropper, and the like. In other cases, the chitosan hydrogels can be included in a formulation for topical administration, for instance, lotions, sprays, creams, ointments and the like. The compositions can also include a backing layer to secure the composition at a wound site.

The pharmaceutical compositions described herein may be prepared by any method known or hereafter developed in the art of pharmaceutics. In general, such preparatory methods include the step of bringing the chitosan hydrogel into association with one or more excipients and/or one or more other accessory ingredients, and then, if necessary and/or desirable, shaping and/or packaging the product into a desired single- or multi-dose unit.

Dosage forms for topical and/or transdermal administration of the chitosan hydrogels may include ointments, pastes, creams, lotions, gels, powders, solutions, sprays, inhalants and/or patches. Generally, the chitosan hydrogel is admixed under sterile conditions with a pharmaceutically acceptable excipient and/or any needed preservatives and/or buffers as may be required. Additionally, the present invention contemplates the use of transdermal patches, which often have the added advantage of providing controlled delivery of an active ingredient to the body. Such dosage forms may be prepared, for example, by dissolving and/or dispensing the chitosan hydrogels in the proper medium. Alternatively or additionally, the rate may be controlled by either providing a rate controlling membrane and/or by dispersing the chitosan hydrogel in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Formulations for topical administration may further comprise one or more of the excipients and/or additional ingredients described herein.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

The chitosan hydrogels may be prepared by complexing chitosan with one or more metal ions in an aqueous mixture. In some implementations, the chitosan hydrogels may be prepared by combining chitosan with one or more metal ions in the presence of ammonia (or ammonia atmosphere), which may be directly supplied as a gas or generated by decomposition of an ammonium salt. In some implementations, the chitosan and metals ions can be combined at room temperature (i.e., about 23° C.) to form the hydrogel. In some implementations, the chitosan and metals ions can be combined at a temperature from 20-30° C., from 25-35° C., from 30-40° C., from 30-50° C., from 30-70° C., from 50-75° C., or from 50-100° C. Once formed, the chitosan hydrogels may be separated from solvents and unreacted reagents by washing/dialyzing with water. The formed chitosan hydrogel mat be combined with additional excipients (e.g., antioxidants, chelating agents, preservatives).

The chitosan, prior to combining with the metal ions, may be provided as an aqueous dispersion or solution at a concentration from 0.1-10 wt. %, from 0.1-5 wt. %, from 0.1-2.5 wt. %, from 0.1-1 wt. %, from 1-10 wt. %, from 1-5 wt. %, from 1-2.5 wt. %, from 2-10 wt. %, from 2-5 wt. %, from 3-10 wt. %, from 3-5 wt. %, or from 5-10 wt. %. Prior to combining with the metal ions, the chitosan may be combined with additional excipients (e.g., antioxidants, chelating agents, preservatives).

In certain implementations, the chitosan that is combined with the metal ions is at least 60% deacylated, at least 65% deacylated, at least 70% deacylated, at least 75% deacylated, at least 80% deacylated, at least 85% deacylated, at least 90% deacylated, at least 95% deacylated, or at least 98% deacylated. In certain implementations, the chitosan that is combined with the metal ions has a degree of diacylation from 60%-98%, from 60%-90%, from 60%-80%, from 60%-70%, from 70%-98%, from 70%-90%, from 70%-80%, 80%-98%, from 80%-90%, or from 90%-98%. In certain implementations, the chitosan that is combined with the metal ions has a degree of N-acylation from 2-40%, from 10%-40%, from 20%-40%, from 20%-30%, from 10%-30%, from 5%-30%, from 5%-20% from 5%-10%, or from 2-5%.

In certain implementations, the chitosan that is combined with the metal ions has an average molecular weight from 500-50,000 Da, from 500-25,000 Da, 500-10,000 Da, from 500-5,000 Da, 500-2,500 Da, from 1,000-25,000 Da, from 1,000-15,000 Da, from 1,000-10,000 Da, from 1,000-5,000 Da, from 1,000-2,500 Da, from 1,500-25,000 Da, from 1,500-10,000 Da, from 1,500-5,000 Da, from 2,500-5,000 Da, from 5,000-15,000, from 5,000-10,000, or from 10,000-25,000 Da.

In some implementations, the metal ions may be provided as water soluble metal salts. Exemplary water-soluble metal salts that may be used to prepare the chitosan hydrogels include AgNO₃, AgF, CuSO₄, CuCl₂, CuNO₃, copper gluconate, FeCl₃, FeCl₂, and combinations thereof. In certain implementations the metal salt is silver nitrate. The metal salts may be provided as aqueous solutions. Prior to combining with the chitosan, the metal salt solution may be combined with additional excipients (e.g., antioxidants, chelating agents, preservatives).

In some implementations the pH of the aqueous solution of chitosan, metal salt, or both is pH adjusted to increase the water solubility of the metal salts and/or chitosan. In some instances, the aqueous solution of the metal salt has a pH from 1-7, from 2-7, from 3-7, from 4-7, from 5-7, from 6-7, from 1-4, from 2-6, from 3-5, from 2-5, from 2-4, or from 3-6. In some implementations, the aqueous solution of the chitosan prior to combining with the metal salt has a pH from 1-7, from 2-7, from 3-7, from 4-7, from 5-7, from 6-7, from 1-4, from 2-6, from 3-5, from 2-5, from 2-4, or from 3-6. The pH may be adjusted using acids such as HCl, HBr, HI, H2SO₄, HNO₃, H₃PO₄, or an organic acid such as acetic acid or formic acid.

In some implementations, the concentration of the aqueous metal salt that is combined with chitosan is from 0.001-3 wt. %, from 0.001-2 wt. %, from 0.001-1 wt. %, from 0.001-1 wt. %, from 0.01-1 wt. %, from 0.1-1 wt. %, from 0.001-0.01 wt. %, from 0.01-0.1 wt. %, from 0.025-0.5 wt. %, from 0.05-0.25 wt. %, from 0.025-0.25 wt. %, from 0.05-0.5 wt. %, from 0.1-0.5 wt. %, from 0.5-1 wt. %, from 0.5-2 wt. %, from 0.5-3 wt. %, from 1-2 wt. %, from 1-3 wt. %, or from 2-3 wt. %.

In some implementations, the chitosan may be combined with the metal salt at a weight ratio (chitosan:metal salt) from 100:1 to 1:1, from 50:1 to 1:1, from 25:1 to 1:1, from 10:1 to 1:1, from 100:1 to 50:1, from 75:1 to 25:1, from 50:1 to 25:1, from 50:1 to 40:1, from 40:1 to 10:1, from 40:1 to 25:1, from 40:1 to 20:1, from 20:1 to 5:1, or from 30:1 to 5:1.

In some implementations, the concentration of the aqueous silver nitrate that is combined with chitosan is from 0.001-3 wt. %, from 0.001-2 wt. %, from 0.001-1 wt. %, from 0.001-1 wt. %, from 0.01-1 wt. %, from 0.1-1 wt. %, from 0.001-0.01 wt. %, from 0.01-0.1 wt. %, from 0.025-0.5 wt. %, from 0.05-0.25 wt. %, from 0.025-0.25 wt. %, from 0.05-0.5 wt. %, from 0.1-0.5 wt. %, from 0.5-1 wt. %, from 0.5-2 wt. %, from 0.5-3 wt. %, from 1-2 wt. %, from 1-3 wt. %, or from 2-3 wt. %.

In some implementations, the chitosan may be combined with silver nitrate at a weight ratio (chitosan:silver salt) from 100:1 to 1:1, from 50:1 to 1:1, from 25:1 to 1:1, from 10:1 to 1:1, from 100:1 to 50:1, from 75:1 to 25:1, from 50:1 to 25:1, from 50:1 to 40:1, from 40:1 to 10:1, from 40:1 to 25:1, from 40:1 to 20:1, from 20:1 to 5:1, or from 30:1 to 5:1.

### EXAMPLES

The following examples are for the purpose of illustration of the invention only and are not intended to limit the scope of the present invention in any manner whatsoever.

### Example 1: Preparation of Chitosan Hydrogel

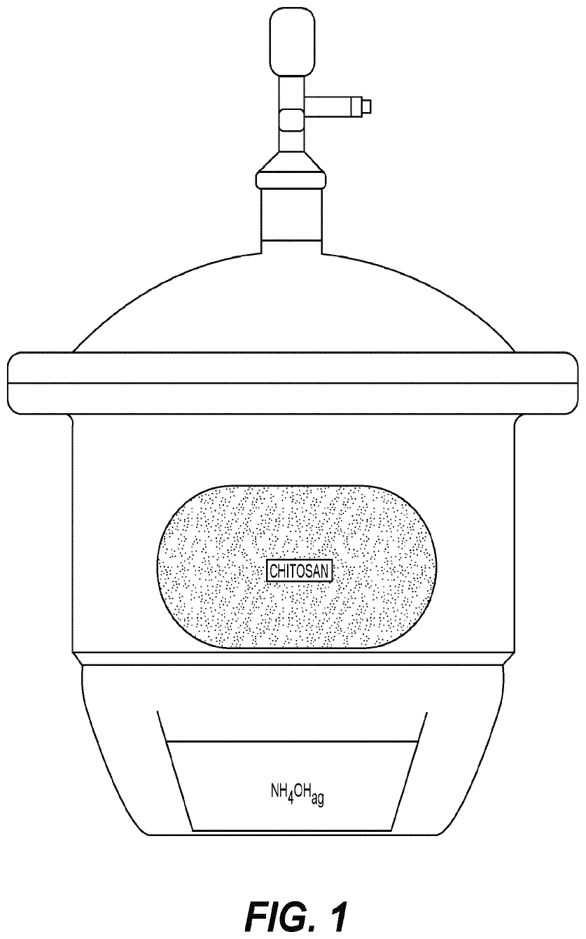

FIG. 1 shows a schematic of the set up for the experiment. Hydrogels were prepared from the polysaccharide chitosan modified by silver (I) ions through complexation interactions. The hydrogels were synthesized using analytical grade chemicals. Chitosan with a deacetylation degree of 85% and an average molecular weight of 1526.464 g mol⁻¹ was obtained from Alfa Aesar™, silver nitrate, glacial acetic acid, and 28.0-30.0 w/w % aqueous ammonium hydroxide was obtained from Fisher Chemical™. The experiments were conducted in deionized water (Milli-Q®, 18.2 MΩ cm⁻¹ at 25° C.).

2 wt. % chitosan was solubilized in 2% (v/v) aqueous acetic acid having varying concentrations of silver nitrate, specifically 0 wt. %, 0.1 wt. %, 0.2 wt. %, and 0.3 wt. % AgNO₃. These amounts correspond to 0.064 wt. %, 0.128 wt. %, and 0.191 wt. % silver ion, respectively. The solutions were prepared in disinfected petri dishes that were placed inside a desiccator. A crystallization disk with ammonia was positioned at the bottom of the desiccator, allowing for slow gelation under a gaseous ammonia atmosphere. After five days, the hydrogels were removed from the desiccator and thoroughly washed with distilled water until reaching a neutral pH value.

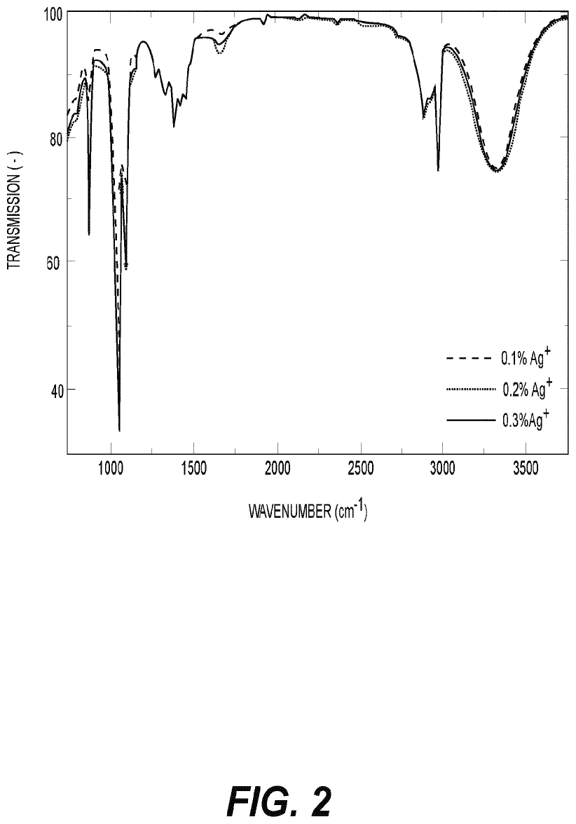

FIG. 2 shows Fourier-Transform Infrared spectrometer (FTIR) spectroscopy of Ag⁺-doped chitosan hydrogels. Chitosan is a polycationic polymer that chelates positively charged, forming a network through ionic bridges. Hydrogel samples were chemically characterized using a Thermo Scientific Nicolet iS10 Fourier-Transform Infrared spectrometer with attenuated total reflection (ATR FTIR) in the range of 400-4000 cm⁻¹ at a resolution of 4 cm⁻¹ and 32 scans. A broad absorption band at ˜3500-3200 cm⁻¹ that was attributed to the stretching vibrations of OH and NH was observed on the spectra of all hydrogels, as shown in FIG. 2. Infrared absorption bands at 1640 cm⁻¹ and 1570 cm⁻¹ belong to the C═O stretching in the amide I and N—H bending vibration in the NH₂ groups of chitosan. Absorption bands at 1410 cm⁻¹, 1151 cm⁻¹, 1092 cm⁻¹ and 1027 cm⁻¹ are associated with C—H symmetrical deformation, the asymmetric stretching of C—O—C bridge, the CO stretching in CH—OH and the C—O stretching in CH₂—OH of chitosan. A higher Ag⁺ content results in a lower ratio between the band intensities at 1570 cm⁻¹ and 1640 cm⁻¹ since the coordinative bonds between —NH₂ moieties and Ag⁺ ions weaken the N—H bending vibration in NH₂ groups.

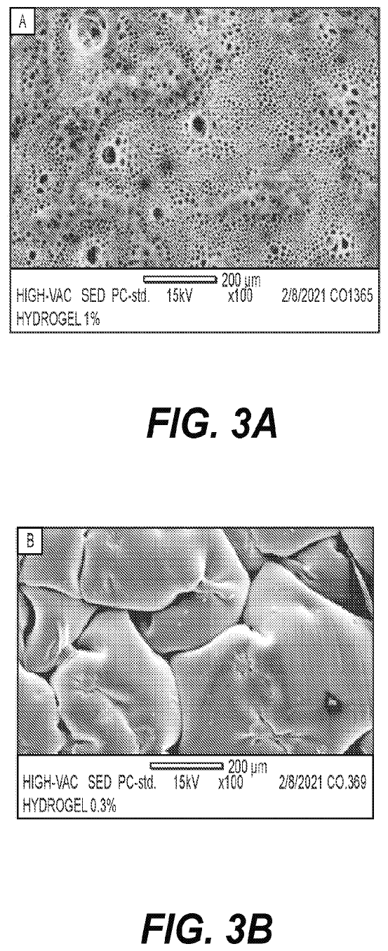

FIGS. 3A and 3B are scanning electron micrographs of silver-doped chitosan hydrogels prepared at 0.1 wt. % and 0.3 wt. % AgNO₃, respectively. Surface morphology and pore size distribution were evaluated using a JEOL Neoscope Scanning Electron Microscopy (SEM). 1 cm3 hydrogel cubes were immersed in a series of alcohol solutions prepared from histological anhydrous ethanol (Fisher Chemical™) of increasing concentrations. The ethanol content was successively increased from 0%, 30%, 70% to 100%. The worked-up hydrogels were dehydrated by critical point drying, mounted on a metal stub with a conductive tape and sputter-coated with a thin gold layer to prevent excessive charging in the SEM. As shown in FIG. 3A, hydrogels prepared at 0.1 wt. % AgNO₃ exhibit a complex pore morphology, while hydrogels prepared at higher silver concentrations have a compact microstructure with folds along the surface (FIG. 3B). We determined the pore size distribution and average pore size using the Dragonfly™ software package. For [0.1 wt. % AgNO₃]hydrogels, we measured an average pore size of 9.57 μm².

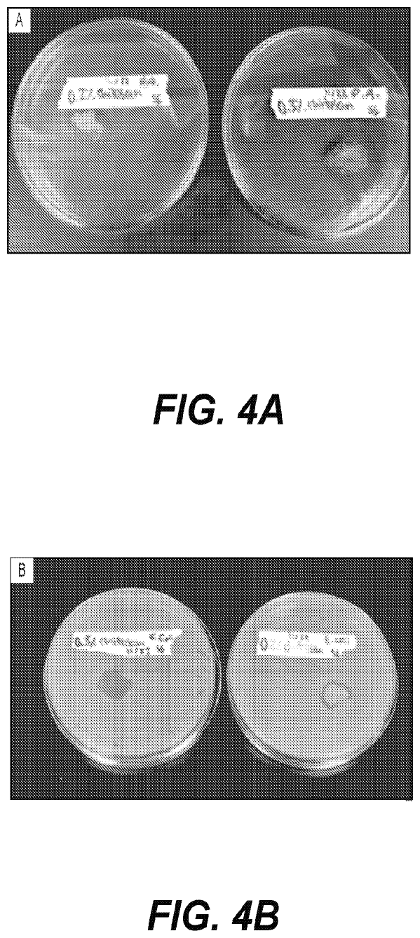

FIG. 4 shows results from Kirby-Bauer tests of silver-doped hydrogels against P. Aeruginosa and E. coli, respectfully, where the results indicate antibacterial properties in the absence of additional antibiotics. To determine whether the hydrogels can be used as wound dressings on their own, we assessed the antibacterial activity against common NI causing bacteria, including Staphylococcus aureus (S. aureus), Pseudomonas aeruginosa (P. aeruginosa), and Escherichia coli (E. coli). S. aureus is a Gram-positive bacterium that is a usual member of the microbiota of the body, frequently found in the upper respiratory tract and on the skin. E. coli is a Gram-negative bacterium that is commonly found in the lower intestine of warm-blooded organisms. P. Aeruginosa is a Gram-negative bacterium that can cause infections in humans, mostly in hospital patients. Hydrogels were cut into small pieces and tested against S. aureus, P. aeruginosa, and E. coli using the Kirby Bauer method (KB). Hydrogels were placed in Mueller-Hinton agar plates that had been inoculated with bacteria cultures. We observed the bacterial inhibition zone by comparing it to the standard inhibition zone. FIG. 4 shows standard inhibition zones that resulted from the preliminary antibacterial tests of the hydrogel [0.1 wt. %, 0.2 wt. %, and 0.3 wt. % AgNO₃] against P. aeruginosa and E. coli in the absence of antibiotics. The lack of bacterial growths around the edges of the gels is consistent with the hydrogel's intrinsic antibacterial activity.

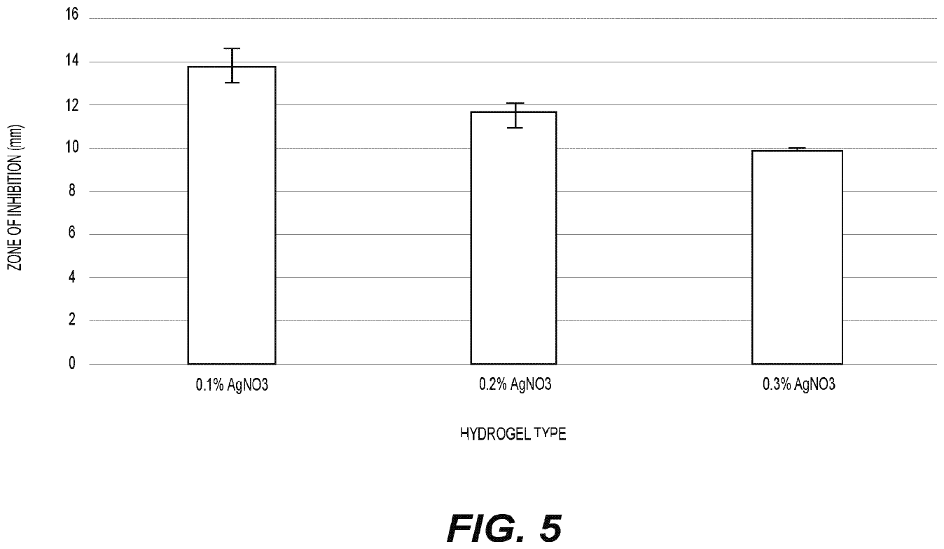

FIG. 5 shows the hydrogel zone of inhibition. The zone of inhibition (representative of antibacterial effect) was measured around the hydrogel. The standard zones of inhibition that resulted from the antibacterial tests of the hydrogels [0.1 wt. %, 0.2 wt. %, and 0.3 wt. % AgNO₃] against S. aureus. ANOVA, Tukey post hoc test, p=0.033, F=13.153.

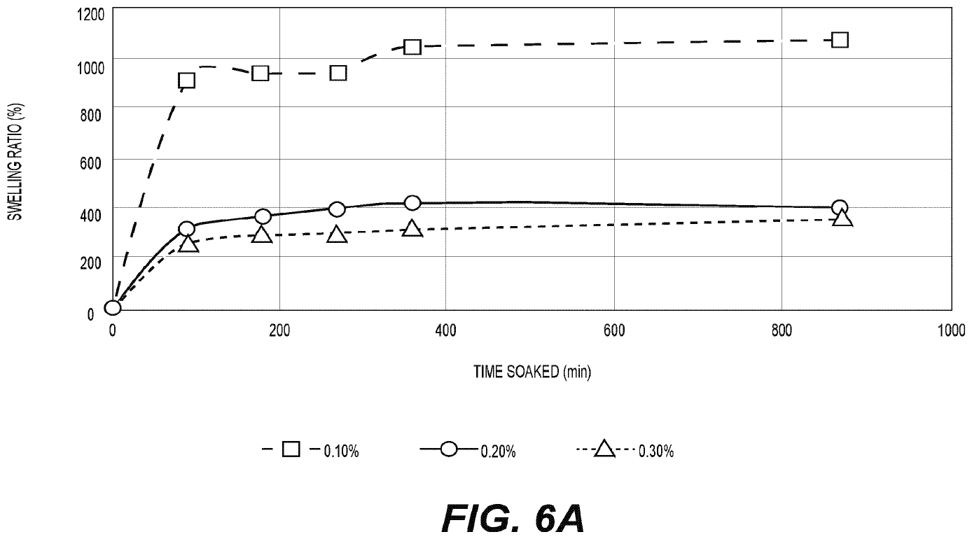

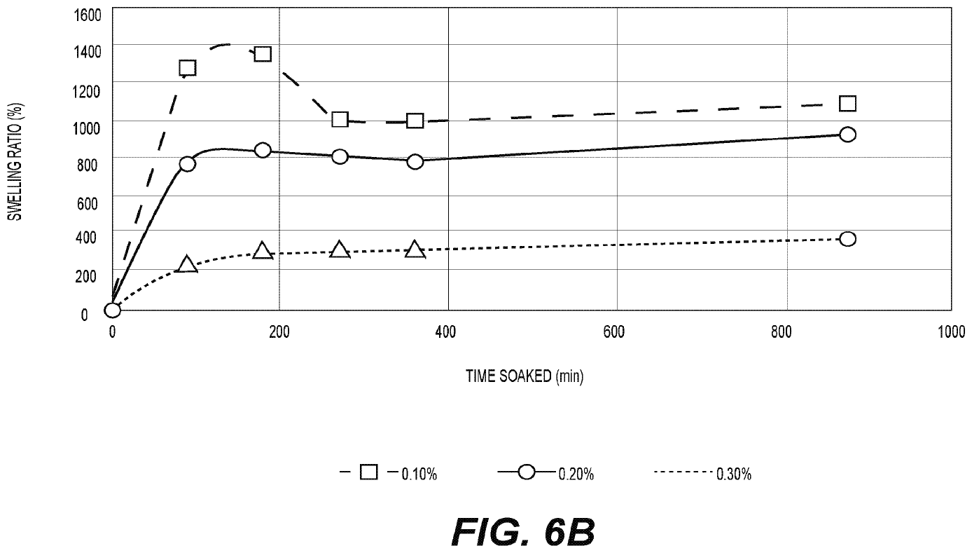

FIGS. 6A and 6B show the swelling ratio of silver-doped hydrogels soaking in deionized water and ethylene glycol, respectfully. Swelling capacity of the hydrogels were evaluated as a function of silver concentration using common solvents, including deionized water (FIG. 6A) and ethylene glycol (FIG. 6B). Water is a common solvent for standard antibiotics, whereas ethylene glycol serves as solvent for novel lipopeptide antibiotics. The data collected suggests a significantly higher swelling ration in [0.1%]hydrogels, which is consistent with the higher porosity observed in SEM.

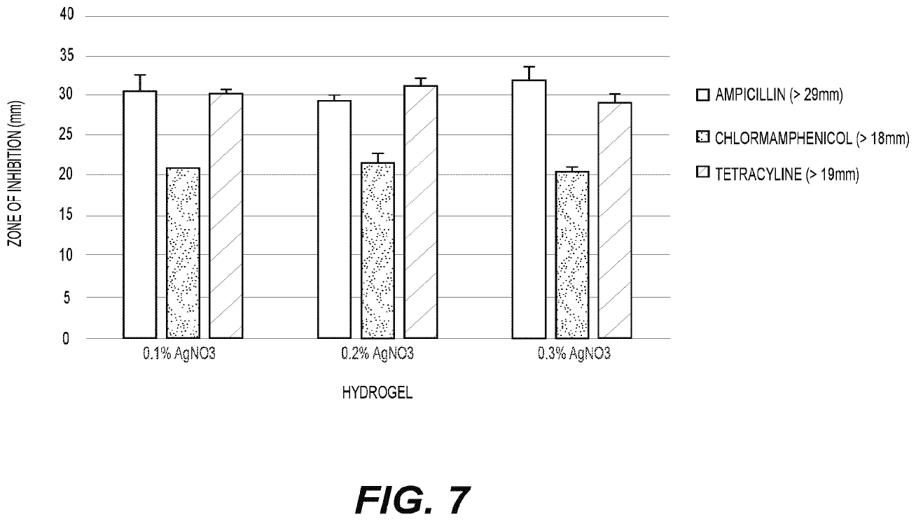

FIG. 7 shows the zones of inhibition that resulted from the antibiotic absorption and release test of the hydrogels [0.1 wt. %, 0.2 wt. %, and 0.3 wt. % AgNO₃] against S. aureus. ANOVA, Tukey post hoc test, p>0.05 for all [AgNO₃] comparisons. Hydrogels were tested via a series of antibiotic absorption and release tests with standard antibiotics, including ampicillin, tetracycline, and chloramphenicol. Hydrogels were left overnight in antibiotic stock solutions and tested against S. aureus using a standard KB test. Zones of inhibition were compared to the standard susceptibility zones that are used to determine effectiveness to the antibiotics in KB tests. The release of the three antibiotics from the hydrogel variations [0.1 wt. %, 0.2 wt. %, and 0.3 wt. % AgNO₃] produced a zone of inhibition that was larger than the susceptibility zone against S. aureus. The silver concentration has no significant effect on the release of antibiotics when the same quantity of antibiotic was loaded into the hydrogel. It was concluded that the concentration of silver nitrate can be used to tailor the absorption properties of the hydrogel, so it could potentially be engineered to release antibiotics at an optimal rate according to patient-specific needs. While further testing is required, this type of product would enable healthcare workers to switch dressings less frequently, decreasing exposure that could lead to NIs, and reduce biohazardous waste.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A biocompatible metal ion-chitosan hydrogel for treating wounds comprising:
chitosan; and
metal ions selected from the group consisting of silver ions, copper ions and combinations thereof;
wherein said hydrogel has antimicrobial properties.

2. The biocompatible metal ion-chitosan hydrogel according to claim 1, wherein the metal ions consists of silver ions.

3. The biocompatible metal ion-chitosan hydrogel according to claim 1, wherein the metal ions consists of copper ions.

4. The biocompatible metal ion-chitosan hydrogel according to claim 1, wherein the metal ions consists of silver ions and copper ions.

5. A method of treating a wound by applying to the wound the biocompatible metal ion-chitosan hydrogel according to claim 1.

6. A method of manufacturing the biocompatible metal ion-chitosan hydrogel according to claim 1 comprising the steps of:
providing the chitosan;
providing the metal ions; and
combining the chitosan and the metal ions to form the biocompatible metal ion-chitosan hydrogel.

7. The biocompatible metal ion-chitosan hydrogel according to claim 2, wherein the hydrogel comprises silver ion at a concentration from 0.001-1 wt. %.

8. The biocompatible metal ion-chitosan hydrogel according to claim 3, wherein the hydrogel comprises copper ion at a concentration from 0.001-1 wt. %.

9. The biocompatible metal ion-chitosan hydrogel according to claim 3, wherein the hydrogel comprises silver ion and copper ions at a combined concentration from 0.001-1 wt. %.

10. The biocompatible metal ion-chitosan hydrogel according to claim 1, wherein the chitosan is at least 60% deacylated.

11. The biocompatible metal ion-chitosan hydrogel according to claim 1, wherein the chitosan has a degree of diacylation from 60%-98%.

12. The biocompatible metal ion-chitosan hydrogel according to claim 1, wherein the chitosan has an average molecular weight from 500-50,000 Da.

13. The biocompatible metal ion-chitosan hydrogel according to claim 1, further comprising at least one additional therapeutic agent.

14. The biocompatible metal ion-chitosan hydrogel according to claim 1, further comprising an analgesic, an anti-inflammatory, an antibiotic, or a combination thereof.

15. The biocompatible metal ion-chitosan hydrogel according to claim 1, further comprising an antibiotic.

16-20. (canceled)

21. The biocompatible metal ion-chitosan hydrogel according to claim 15, wherein the antibiotic comprises CLP-4.

22. A method of treating a wound, comprising contacting the wound with the biocompatible metal ion-chitosan hydrogel according to claim 1.

23. (canceled)

24. The method of claim 22, wherein the biocompatible metal ion-chitosan hydrogel is topically applied to the wound.

25. A pharmaceutical composition comprising the biocompatible metal ion-chitosan hydrogel according to claim 1, and at least one pharmaceutically acceptable excipient.

26. The pharmaceutical composition of claim 25, wherein the composition is a solution, suspension, lotion, creams, or ointment.

27. (canceled)
